(19) United States
(12) Patent Application Publication
(10) Pub. No.: US 2003/0123609 A1
(43) Pub. Date: Jul. 3, 2003

(54) Fiduciary tray for an IMRT collimator

(76) Inventor: Maria A. Manske, Missouri City, TX (US)

Correspondence Address: FULLBRIGHT & JAWORSKI L.L.P., 600 CONGRESS AVE., SUITE 2400, AUSTIN, TX, 78701, US

(21) Appl. No.: 10/310,694

(22) Filed: Dec. 5, 2002

Related U.S. Application Data
(60) Provisional application No. 60/337,619, filed on Dec. 5, 2001.

(51) Int. Cl. A61N 5/10

(52) U.S. Cl. 378/65

(57) ABSTRACT

A fiduciary tray for use in Intensity Modulated Radiation Therapy (IMRT). In an exemplary embodiment the tray includes a first horizontal surface, a second horizontal surface, vertical walls, and a plurality of openings. The second horizontal surface is located above the first horizontal surface and is configured to enter an opening of a collimator. The vertical walls couple the first and second horizontal surfaces and engage a rim within the opening of the collimator. The plurality of openings in the second horizontal surface accept radiopaque materials.

This application claims priority to, and incorporates by reference, U.S. Provisional Patent Application Serial No. 60/337,619, which was filed on Dec. 5, 2001 by Maria A. Manske and which was also entitled, “Fiduciary Tray for an IMRT Collimator.”

### BACKGROUND OF THE INVENTION

1. Field of the Invention

The present invention relates generally to radiation therapy. More particularly, the invention relates to a fiduciary tray that can be used with Intensity Modulated Radiation Therapy (IMRT).

2. Background

IMRT is an approach to conformal therapy that not only conforms dose to the target volume, but also conforms dose away from sensitive structures. Conformal therapy typically shapes a treatment beam so that its contour corresponds to a beam's eye view of a target plus margin.

IMRT allows specific modifications to be made to dose distribution by controlling the movement of shutters or leaves in a collimator. Even more control is gained when the beam is allowed to move in an arc or other pattern around a patient. Through the use of collimators and movement, IMRT is able to deliver nonuniform radiation exposure to the patient to create a uniform dose distribution at a target site. Effectively, the target site is exposed to a certain extent while sensitive structures of the patient are exposed to a lesser extent.

Although IMRT represents an improvement over previous radiation therapy techniques, shortcomings remain. In particular, until now, there has been no fiduciary tray designed for use with an IMRT device. As will be described in more detail below, such a fiduciary tray allows the practitioner to better evaluate proper patient alignment prior to exposure, which is becoming more and more important with the advent of conformal treatment and the escalation of dose.

Accordingly, a significant need exists for the techniques described and claimed in this disclosure.

### SUMMARY OF THE INVENTION

Particular shortcomings of the prior art are reduced or eliminated by the techniques discussed in this disclosure. Specifically, this disclosure describes suitable fiduciary trays that allow for better anatomy-localization and which greatly aid in the interpretation of port verification films.

In one respect, the invention involves a fiduciary tray for use in Intensity Modulated Radiation Therapy (IMRT), including a first horizontal surface, a second horizontal surface, vertical walls, and a plurality of openings. The second horizontal surface is located above the first horizontal surface and is configured to enter an opening of a collimator. The vertical walls couple the first and second horizontal surfaces and engage a rim within the opening of the collimator. The plurality of openings in the second horizontal accept radiopaque materials.

In other respects, the plurality of openings may be spaced to cast shadows separated by a pre-determined distance on a port film. The plurality of openings may be arranged along a centerline parallel to a width of the tray and along a centerline parallel to a length of the tray. The plurality of openings may be spaced to cast shadows 1 cm apart. The tray may also include holes configured to receiving mounting bolts. The may also include a lockout component configured to project into the opening of the collimator to impede collimator leaves.

In another respect, the invention involves a fiduciary tray for use in Intensity Modulated Radiation Therapy (IMRT), including a body, a first horizontal surface, a second horizontal surface, vertical walls, a first plurality of openings, a second plurality of openings, and means for preventing accidental treatment. The body has a width and length. The second horizontal surface is located above the first horizontal surface and is configured to enter an opening of a collimator. The vertical walls couple the first and second horizontal surfaces and engage a rim within the opening of the collimator. The first plurality of openings are in the second horizontal surface and accept radiopaque materials. The first plurality of openings are arranged along a centerline parallel to the width the body and are spaced to cast shadows separated by a pre-determined distance on a port film. The second plurality of openings are in the second horizontal surface and accept radiopaque materials. The second plurality of openings are arranged along a centerline parallel to the length of the body and are spaced to cast shadows separated by a pre-determined distance on a port film. The means for preventing accidental treatment ensure that a patient is not treated while the tray is in use.

In other respects, the tray may also include holes configured to receiving mounting bolts. The means for preventing accidental treatment may include an apparatus configured to detect the presence of the mounting bolts. The means for preventing accidental treatment may alternatively include a lockout component configured to project into the opening of the collimator to impede collimator leaves.

In another respect, the invention involves an Intensity Modulated Radiation Therapy (IMRT) system including a fiduciary tray, including an accelerator, a head coupled to the accelerator, a collimator coupled to the head, and a fiduciary tray as described above.

In another respect, the invention involves a method of Intensity Modulated Radiation Therapy (IMRT). One first obtains a fiduciary tray as described above. Then, a port film is exposed using that fiduciary tray.

As used herein, “a” and “an” shall not be strictly interpreted as meaning “one” unless the context of the invention necessarily and absolutely requires such interpretation.

Other features and associated advantages will become apparent with reference to the following detailed description of specific embodiments in connection with the accompanying drawings.

### BRIEF DESCRIPTION OF THE DRAWINGS

The techniques of this disclosure may be better understood by reference to one or more of these drawings in combination with the detailed description of illustrative embodiments presented herein. Identical or similar elements use the same element number. The drawings are not necessarily drawn to scale.

FIG. 1 is a perspective view of a fiduciary tray in accordance with embodiments of the present disclosure.

FIG. 2 is a perspective view of a linear accelerator with an IMRT collimator installed.

FIG. 3 is a partially-exploded, perspective view of an IMRT collimator mounted on the head of the linear accelerator of FIG. 2 and of the fiduciary tray of FIG. 1, the system being in accordance with embodiments of the present disclosure.

FIG. 4 is a perspective view of a fiduciary tray including a lockout component, in accordance with embodiments of the present disclosure.

### DESCRIPTION OF ILLUSTRATIVE EMBODIMENTS

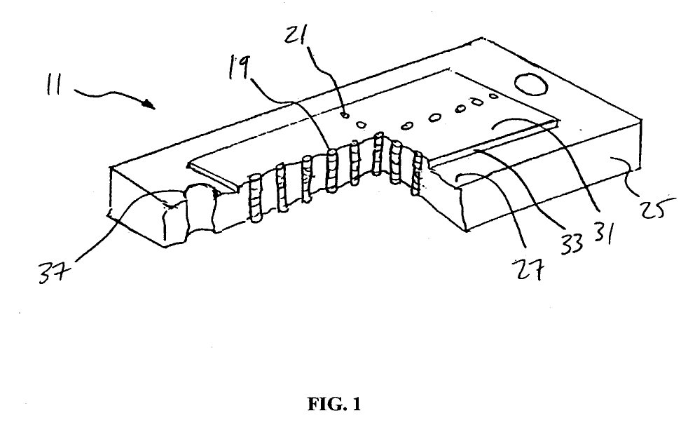

FIGS. 1 through 4 show a fiduciary, or shadow, tray 11, a linear accelerator 13, an IMRT collimator 15, and a lockout component 48 for use with tray 11, respectively. In illustrated embodiments, collimator 15 is a MIMiC unit, manufactured by NOMOS Corp. of Sewickley, Pa., and is mounted to the head 16 of linear accelerator 13. However, other collimators and devices may be used in practicing this invention.

Collimator 15 allows a narrow, fan-shaped slice of radiation to be emitted towards a patient located on bed 17. Because of the high radiation doses involved, it is necessary to position patients as accurately as possible for each treatment. Tray 11 provides a means of accurately and repeatably positioning patients under collimator 15 by locating radiopaque markers 19, 21 in relation to opening 23 in collimator 15, markers 19, 21 casting shadows when a film (not shown) located in bed 17 and below the patient is exposed through the treatment portal, or port, to radiation passing through tray 11 and through patient. As illustrated, tray 11 is specifically designed to be used on MIMiC collimators, but it will be understood that it may be readily adapted for use with other IMRT devices as well.

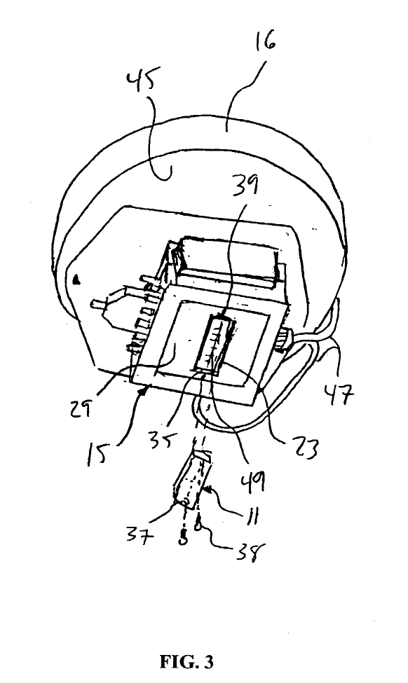

Referring to FIG. 1, tray 11 comprises a body 25 that is preferably formed from a thin, planar portion of clear acrylic, though tray 11 may also be formed from other materials that allow visible light and x-rays to pass through tray 11. Because tray 11 will be mounted and removed repeatedly, body 25 should be formed from materials that also have high strength and that resist scratching. The transmission of visible light is necessary to accommodate a light field (not shown) projected onto the patient using a lamp within collimator 15 (FIG. 3). The light field is used in conjunction with lasers (not shown) mounted in the treatment room to align the patient before treatment begins and before exposing the port film to confirm alignment. Port films are generally taken once a week during treatment. Because tray 11 is removed before treatment doses are applied, attenuation of the x-rays is acceptable. For example, the attenuation of radiation from the acrylic used to form tray 11 is approximately 5%. Because of the high energy levels of the radiation, refraction within tray 11 is minimal.

Body 25 has an horizontal upper surface 27 that is placed against a lower surface 29 (FIG. 3) of collimator 15 when tray 11 is mounted on collimator 15. A second horizontal surface 31 is located above surface 29 and is separated from surface 29 by vertical walls 33 forming the perimeter of surface 31. Walls 33 are sized for engaging rim 35 (FIG. 3) located above surface 29 and within opening 23 when tray 11 is mounted to collimator 15. Holes 37 are located at each end of tray 11 for receiving mounting bolts 38 to mount tray 11 to collimator 15 at mounting holes 39 (FIG. 3).

Markers 19, 21 are cylindrical and are preferably formed from Tungsten rod stock, though other radiopaque materials may be used, providing that the other materials produce a similarly sharp film image. Markers 19 are aligned along a centerline that is parallel to the width of body 25, whereas markers 21 are aligned along a centerline that is parallel to the length of body 25. Markers 19, 21 are inserted into tray 11 with the axes of markers 19, 21 being generally vertical, markers 19, 21 preferably extending through the entire height of tray 11. Because the distance from tray 11 to the patient is known, markers 19, 21 can be inserted into tray 11 with distances between them that produce a pre-determined distance between the shadows cast on the port films, the shadows corresponding to a known length at a level within the patient. For example, markers 19, 21 in tray 11 may produce shadows that indicate 1 cm increments in length at the level within the patient, though the shadows are more than 1 cm apart from each other on the port film due to magnification. The port films can then be directly compared to the results of other imaging methods, for example, digitally-reconstructed radiographs (DRRs) constructed from computerized tomography (CT) images. Additional markers (not shown) may be added to tray 11 to indicate right and left sides or anterior and posterior directions on the port films.

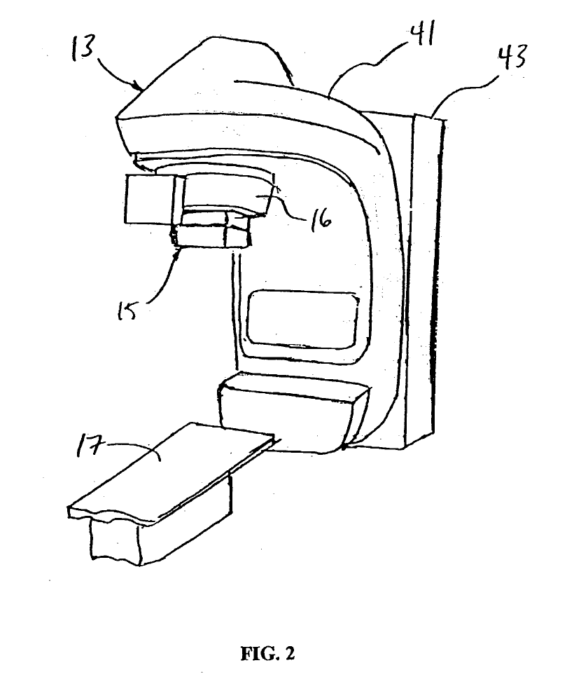

Linear accelerator 13 is illustrated in FIG. 2. Accelerator 13 has a rotating section 41 that is mounted to stationary housing 43. Radiation is produced within accelerator 13 and is directed through section 41 to head 16. Head 16 depends from an inner surface of section 41, and collimator 15 is mounted to head 16. Section 41 rotates on a horizontal axis to move head 16 and collimator 15 in a circular motion around a patient located on bed 17. Additionally, head 16 rotates on an axis to rotate collimator 15, the axis intersects the rotation axis of section 41 throughout rotation of section 41. When section 41 is in the vertical position, as shown in FIG. 2, the port films will image tissues extending from right to left, whereas section 41 can be rotated to the left or right to image tissues extending from the anterior to the posterior regions.

FIG. 3 is a partially-exploded view from beneath collimator 15 mounted to head 16. An opening (not shown) in head 16 allows radiation to pass out of section 41 and through head 16, and collimator 15 is mounted to a lower surface 45 of head 16 and aligned with the opening. Cables 47 connect collimator to control and monitoring systems for use by the operator. Opening 23 surrounds a set of leaves 49 that are used to control the release of radiation from collimator 15, and rim 35 sits above lower surface 29 and within opening 23. Rim 35 has smaller horizontal dimensions and encloses a smaller area than opening 23, allowing walls 33 of tray 11 to extend through opening 23 and to fit snugly within rim 35, ensuring that tray 11 is properly aligned to opening 23. Rim 35 provides for accurate positioning when mounting tray 11, the variation being less than 1 mm.

Referring still to FIG. 3, tray 11 is mounted by moving tray 11 upward until upper surface 27 of tray 11 contacts lower surface 29 of collimator 15. As upper surface 27 approaches lower surface 29, horizontal surface 31 and walls 33 enter opening 23 and rim 35, walls 33 snugly engaging the interior of rim 35. Mounting bolts 38 are inserted through holes 37 of tray 11 and are secured in mounting holes 39 of collimator 15, bolts 38 preferably being captured within holes 37. Linear accelerator 13 and collimator 15 may then be used to expose a port film to determine proper positioning of the patient.

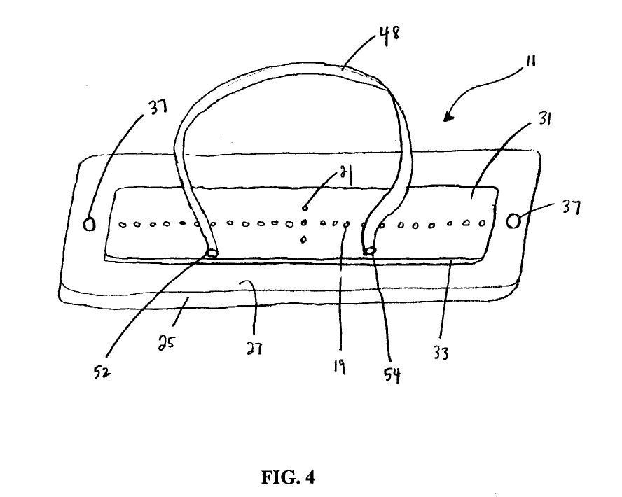

A form of lockout is recommended to prevent accidental treatment of a patient while tray 11 is installed on collimator 15, which would lead to underdosing due to the attenuation of the radiation. A preferred lockout system is one in which the presence of mounting bolts 38 within mounting holes 39 would be detected, the operator being alerted that tray 11 is installed. Alternatively, a simple lockout can be accomplished by providing a component 48 (FIG. 4) that protrudes from horizontal surface 31 and enters leaves 49, engaging a system that alerts the operator that tray 11 is installed by producing a default in the software. In FIG. 4, lockout component 48 is a plastic extension that is looped through two holes 52, 54 in tray 11. It is centered such that it projects into opening 23 far enough to impede leaves 49. Being made of flexible plastic, or a similar flexible material, leaves 49 are not damaged by the presence of lockout component 48.

With the benefit of the present disclosure, those having skill in the art will comprehend that techniques claimed herein and described above may be modified and applied to a number of additional, different applications, achieving the same or a similar result. For example, the fiduciary tray illustrated herein may be modified to fit collimators from other manufacturers or for different size fields. The claims cover all modifications that fall within the scope and spirit of this disclosure.

What is claimed is:

1. A fiduciary tray for use in Intensity Modulated Radiation Therapy (IMRT), the tray comprising:
a first horizontal surface;
a second horizontal surface located above the first horizontal surface, the second horizontal surface configured to enter an opening of a collimator;
vertical walls coupling the first and second horizontal surfaces, the vertical walls configured to engage a rim within the opening of the collimator; and
a plurality of openings in the second horizontal surface configured to accept radiopaque materials.

2. The tray of claim 1, the plurality of openings being spaced to cast shadows separated by a pre-determined distance on a port film.

3. The tray of claim 2, the plurality of openings being arranged along a centerline parallel to a width of the tray and along a centerline parallel to a length of the tray.

4. The tray of claim 3, the plurality of openings being spaced to cast shadows 1 cm apart.

5. The tray of claim 1, further comprising holes configured to receiving mounting bolts.

6. The tray of claim 1, further comprising a lockout component configured to project into the opening of the collimator to impede collimator leaves.

7. A fiduciary tray for use in Intensity Modulated Radiation Therapy (IMRT), the tray comprising:
a body having a width and length;
a first horizontal surface;
a second horizontal surface located above the first horizontal surface, the second horizontal surface configured to enter an opening of a collimator;
vertical walls coupling the first and second horizontal surfaces, the vertical walls configured to engage a rim within the opening of the collimator;
a first plurality of openings in the second horizontal surface configured to accept radiopaque materials, the first plurality of openings being arranged along a centerline parallel to the width the body and being spaced to cast shadows separated by a pre-determined distance on a port film;
a second plurality of openings in the second horizontal surface configured to accept radiopaque materials, the second plurality of openings being arranged along a centerline parallel to the length of the body and being spaced to cast shadows separated by a pre-determined distance on a port film; and
means for preventing accidental treatment of a patient while the tray is in use.

8. The tray of claim 7, further comprising holes configured to receiving mounting bolts.

9. The tray of claim 8, the means comprising an apparatus configured to detect the presence of the mounting bolts.

10. The tray of claim 7, the means comprising a lockout component configured to project into the opening of the collimator to impede collimator leaves.

11. An Intensity Modulated Radiation Therapy (IMRT) system including a fiduciary tray, comprising:
an accelerator;
a head coupled to the accelerator;
a collimator coupled to the head, the collimator comprising an opening including a rim; and
a fiduciary tray configured couple to the collimator, the fiduciary tray comprising:
a first horizontal surface;
a second horizontal surface located above the first horizontal surface, the second horizontal surface configured to enter the opening of the collimator;
vertical walls coupling the first and second horizontal surfaces, the vertical walls configured to engage the rim; and
a plurality of openings in the second horizontal surface configured to accept radiopaque materials.

12. A method of Intensity Modulated Radiation Therapy (IMRT), the method comprising:
obtaining a fiduciary tray comprising:
a first horizontal surface;
a second horizontal surface located above the first horizontal surface, the second horizontal surface configured to enter the opening of the collimator;
vertical walls coupling the first and second horizontal surfaces, the vertical walls configured to engage the rim; and
a plurality of openings in the second horizontal surface configured to accept radiopaque materials; and
exposing a port film using the fiduciary tray.
